(19) United States
(12) Patent Application Publication
(10) Pub. No.: US 2013/0284196 A1
(43) Pub. Date: Oct. 31, 2013

(54) Applicator Assembly for Applying a Composition

(71) Applicant: THE PROCTER & GAMBLE COMPANY, Cincinnati, OH (US)

(72) Inventors: William Dale MURDOCK, Cheviot, OH (US); Mikah COFFINDAFFER, Mason, OH (US); James Douglas STILL, Cleves, OH (US); Joseph Allen BERLEPSCH, Hamilton, OH (US)

(73) Assignee: The Procter & Gamble Company, Cincinnati, OH (US)

(21) Appl. No.: 13/871,231

(22) Filed: Apr. 26, 2013

Related U.S. Application Data
(60) Provisional application No. 61/639,462, filed on Apr. 27, 2012.

(51) Int. Cl. A45D 44/00

(52) U.S. Cl. CPC A45D 44/00
USPC 132/200; 401/28

(57) ABSTRACT

A method for delivering a composition to the scalp including (a) providing a composition in an applicator assembly, the applicator assembly including (i) a container for holding the composition; (ii) an extended tip actuator in fluid communication with the container, the extended tip actuator including (1) a base portion configured to fluidly connect the extended tip actuator to the container; and (2) a body portion configured to fluidly connect the base portion to a plurality of hollow tines; and (iii) an engine for delivering the composition from the container through the extended tip actuator; and (b) dispensing the composition from the applicator assembly directly onto the scalp. The tines each include a face located distally from the body portion. The tines each include an aperture in fluid communication with the container. The tines each have a protrusion length of from about 0.5 mm to about 100 mm.

### FIELD OF THE INVENTION

The present invention relates to an applicator assembly and a method for applying a composition. More particularly, the invention relates to an applicator assembly having a container, an extended multi-tip actuator with a plurality of hollow tines, and an engine for applying a composition, and methods thereof.

### BACKGROUND OF THE INVENTION

Various devices have been made for applying compositions to the scalp. Such devices have been used to apply compositions for the purposes of conditioning, cleansing, dying, and/or applying one or more benefit agents. However, the designs of currently marketed devices can lead to consumer confusion as to how to use the device, resulting in decreased efficacy and inefficient delivery of the composition.

Based on the foregoing, there is a need for a unique delivery system which effectively communicates to the consumer how to use the product, improves the efficacy of the composition applied, and delivers optimal dosage of the composition to the scalp.

### SUMMARY OF THE INVENTION

According to an embodiment of the invention, there is provided a method for delivering a composition to the scalp comprising: (a) providing a composition in an applicator assembly, the applicator assembly comprising: (i) a container for holding the composition; (ii) an extended tip actuator in fluid communication with the container, the extended tip actuator comprising: (1) a base portion configured to fluidly connect the extended tip actuator to the container; and (2) a body portion configured to fluidly connect the base portion to a plurality of hollow tines, wherein the tines each comprise a face located distally from the body portion, wherein the tines each comprise an aperture in fluid communication with the container, and wherein the tines each have a protrusion length of from about 0.5 mm to about 100 mm; and (iii) an engine for delivering the composition from the container through the extended tip actuator; and (b) dispensing the composition from the applicator assembly directly onto the scalp.

According to yet another embodiment of the invention, there is provided an applicator assembly for delivering a composition to the scalp comprising: (a) a container for holding the composition; (b) an extended multi-tip actuator in fluid communication with the container, the extended tip actuator comprising: (i) a base portion configured to fluidly connect the extended multi-tip actuator to the container; and (ii) a body portion configured to fluidly connect the base portion to a plurality of hollow tines, wherein the tines each comprise a face located distally from the body portion, wherein the tines each comprise an aperture in fluid communication with the container, and wherein the tines each have a protrusion length of from about 0.5 mm to about 100 mm; and (c) an engine for delivering the composition from the container through the extended multi-tip actuator to the scalp.

These and other features, aspects, and advantages of the present invention will become evident to those skilled in the art from a reading of the present disclosure.

### BRIEF DESCRIPTION OF THE DRAWINGS

While the specification concludes with the claims particularly pointing out and distinctly claiming the invention, it is believed that the present invention will be better understood from the following description taken in conjunction with the accompanying drawings in which:

FIG. 1 is a perspective front view of one embodiment of the applicator assembly;

FIG. 2 is a front view of the applicator assembly of FIG. 1;

FIG. 2A is an enlarged front view of the extended multi-tip actuator, taken of the area included in circle 2A in FIG. 2;

FIG. 3 is a top view of the applicator assembly of FIG. 1;

FIG. 4 is a right side view of the applicator assembly of FIG. 1; and

FIG. 5 is a left side view of another embodiment of the applicator assembly.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

The term “comprising,” as used herein, means that other steps and other ingredients which do not affect the end result can be added. This term encompasses the terms “consisting of” and “consisting essentially of.” The compositions and methods/processes of the present invention can comprise, consist of, and consist essentially of the elements and limitations of the invention described herein, as well as any of the additional or optional ingredients, components, steps, or limitations described herein.

The terms “include,” “includes,” and “including,” as used herein, are meant to be non-limiting and are understood to mean “comprise,” “comprises,” and “comprising,” respectively. The term “scalp,” as used herein, includes the roots of the hair.

The test methods disclosed in the Test Methods Section of the present application should be used to determine the respective values of the parameters of Applicants' inventions.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated. The term “weight percent” may be denoted as “wt. %” herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### A. Container

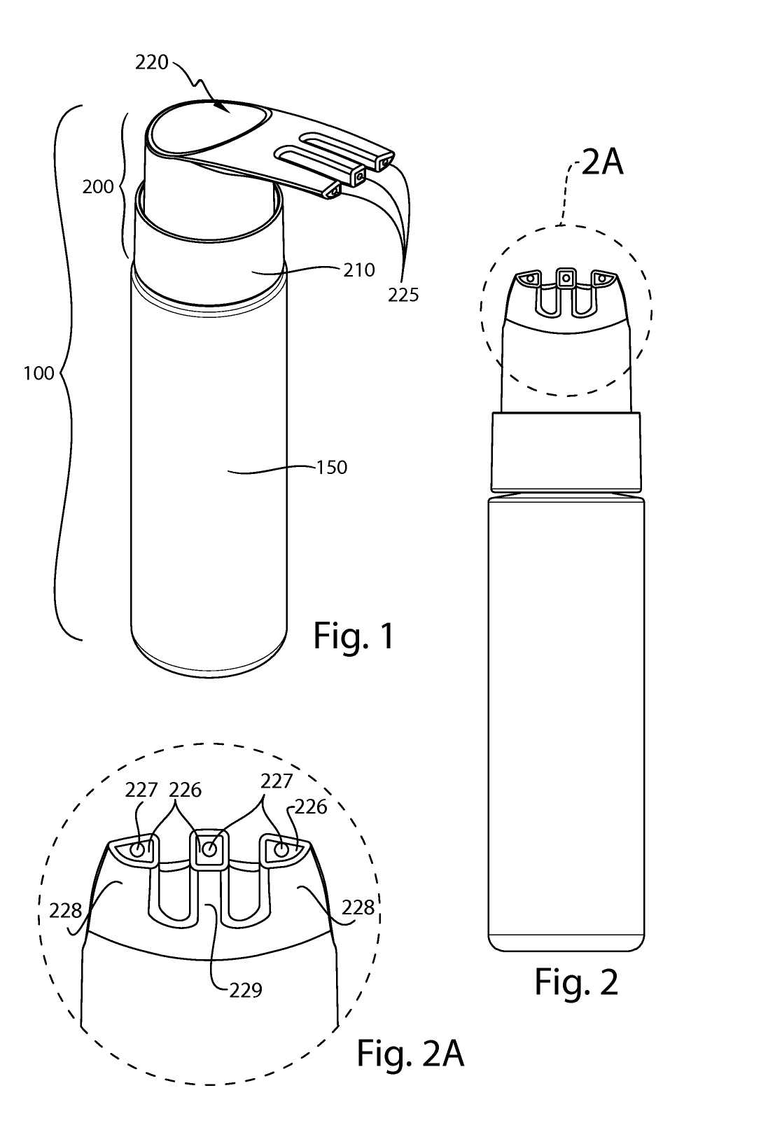

Referring to FIG. 1, the applicator assembly 100 may comprise a container 150. The container 150 may be of any type that is suitable for holding a composition. In one embodiment, the container 150 may be substantially rigid. The container 150 is substantially rigid if it does not collapse under external atmospheric pressure when it is subject to an interior partial vacuum. In an embodiment, the container 150 may comprise a non-rigid material. The non-rigid material may be designed for being squeezed.

The container 150 may be made out of any suitable material selected from the group consisting of plastic, metal, alloy, laminate, and combinations thereof. The container 150 may be any shape that fits the holding structure and may comprise at least one interior compartment for containing at least one fluid. The container 150 may be a refillable container such as a pour-in or screw-on container, or the container 150 may be for one-time use. The container 150 may also be removable from the applicator assembly 100. Alternatively, the container 150 may be integrated with applicator assembly 100.

### B. Extended Multi-Tip Actuator

Still referring to FIG. 1, the applicator assembly 100 may comprise an extended multi-tip actuator 200. The extended multi-tip actuator 200 may be in fluid communication with the container 150. The extended multi-tip actuator 200 may comprise a base portion 210, a body portion 220, and a plurality of hollow tines 225. The extended multi-tip actuator 200 may be made out of any suitable material selected from the group consisting of plastic, metal, alloy, and combinations thereof. The extended multi-tip actuator 200 may be removable from the applicator assembly 100. Alternatively, the extended multi-tip actuator 200 may be integrated with the applicator assembly 100.

### 1. Base Portion

Referring to FIG. 1, the extended multi-tip actuator 200 may comprise a base portion 210.

The base portion 210 may be configured to fluidly connect the extended multi-tip actuator 200 to the container 150. The base portion 210 may be removable from the extended multi-tip actuator 200. Alternatively, the base portion 210 may be integrated with the extended multi-tip actuator 200.

### 2. Body Portion

Still referring to FIG. 1, the extended multi-tip actuator 200 may comprise a body portion 220. The body portion 220 may be configured to fluidly connect the base portion 210 to a plurality of hollow tines 225. The body portion 220 may be removable from the extended multi-tip actuator 200. Alternatively, the body portion 210 may be integrated with the extended multi-tip actuator 200. The multi-tip actuator 200 has been found to have improved usefulness for scalp application, improved ease of application, and improved efficiency of application versus a both single tine actuators and/or common spray actuators used for dispensing compositions onto the scalp.

### 3. Plurality of Hollow Tines

Still referring to FIG. 1, the extended multi-tip actuator 200 may comprise a plurality of hollow tines 225. The plurality of hollow tines 225 may each comprise a face located distally from the body portion 220. The tines may also comprise an aperture in fluid communication with the container 150. The tines may be removable from the extended multi-tip actuator 200. Alternatively, the tines may be integrated with the extended multi-tip actuator 200.

In one embodiment, the flow rate is balanced across the plurality of hollow tines 225 when the applicator assembly 100 comprises a composition. The flow rate can be balanced in multiple ways, including but not limited to, a modification to the inside diameter of the tines, location of the flow channel(s) into the tines from the engine and/or a collection-distribution manifold, and incorporation of internal baffles to impact the fluid flow.

The fluid flow channels connecting the applicator assembly 100 to the outlet of the tines may impact the pressure drop and therefore the fluid flow rate through each tine. Changes to the tine diameter, cross sectional shape and area, and overall flow path protrusion length may induce changes in flow rate through the individual tines. The rheology of the fluid being dispensed may also impact the balance of flow across the tines.

In complex rheology fluids, such as shear thinning fluids, the apparent viscosity changes with shear rate through the flow path. Smaller cross sectional area and higher flow rates will tend to give higher shear rates. The differences in shear rates may then induce differences in apparent viscosity, making design of the flow path to balance flow rate across tines more challenging. Design of systems with balanced flow across tines may be accomplished by building experimental prototypes and using numerical simulations that involve computational fluid dynamics

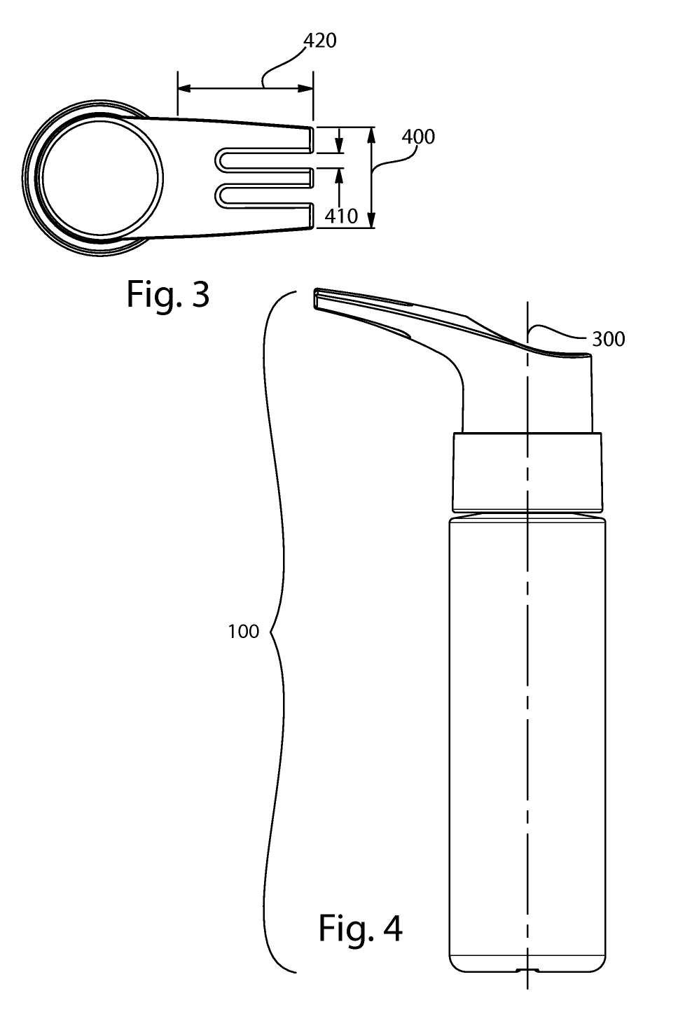

Now referring to FIG. 3, the plurality of hollow tines 225 may each have a protrusion length 420. The protrusion length 420 is the distance that one or more of the plurality of hollow tines 225 protrudes from the outer contours of the container 150. In one embodiment, one or more of the plurality of hollow tines 225 each may have a protrusion length 420 of from about 0.5 mm to about 100 mm, alternatively from about 5 mm to about 80 mm, alternatively from about 10 mm to about 60 mm, alternatively from about 15 mm to about 55 mm, alternatively from about 20 mm to about 50 mm, and alternatively from about 25 mm to about 45 mm. In another embodiment, the plurality of hollow tines 225 each may have a protrusion length 420 of from about 0.5 mm to about 30 mm, and alternatively from about 10 mm to about 25 mm.

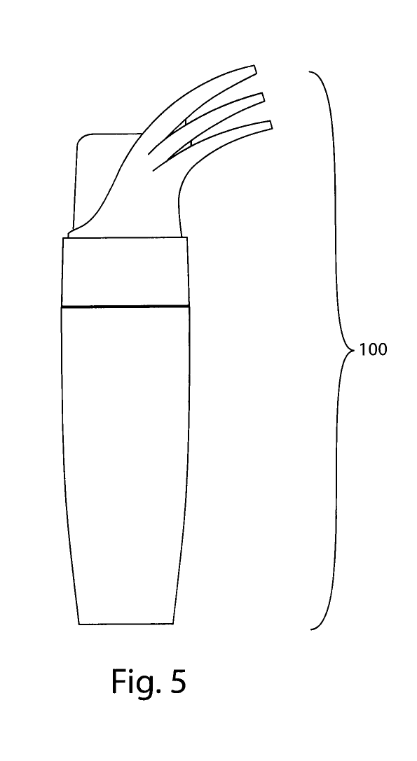

The plurality of hollow tines 225 may comprise at least two tines. The tines may be arranged linearly or staggered in different rows. In one embodiment, the plurality of hollow tines 225 may comprise from two to twelve tines. In another embodiment, the plurality of hollow tines may comprise two to five tines. In yet another embodiment, the plurality of hollow tines may comprise three tines. In an embodiment, the tines may be positioned vertically, as shown in FIG. 5. In an embodiment, the tines may be positioned horizontally. In an embodiment, one or more of the tines may have a different length than the other tines.

When the plurality of hollow tines 225 comprises three tines, the three tines may comprise two external tines 228 and one internal tine 229, wherein the diameter of the aperture 227 of the internal tine 229 is from about 1% to about 40% greater than the diameter of the apertures 227 of the two external tines 228, alternatively the diameter of the aperture 227 of the internal tine 229 is from about 2% to about 20% greater than the diameter of the apertures 227 of the two external tines 228, alternatively the diameter of the aperture of the internal tine 229 is from about 5% to about 15% greater than the diameter of the apertures 227 of the two external tines 228, and alternatively the diameter of the aperture of the internal tine 229 is from about 8% to about 12% greater than the diameter of the apertures 227 of the two external tines 228.

Referring to FIG. 4, the applicator assembly 100 may further comprise a longitudinal axis 300 therethrough. In one embodiment, the plurality of hollow tines 225 may be angled from about 20° to about 100° from the longitudinal axis 300, alternatively from about 25° to about 70° from the longitudinal axis 300, alternatively from about 30° to about 60° from the longitudinal axis 300, alternatively from about 35° to about 55° from the longitudinal axis 300, and alternatively at about 45° from the longitudinal axis 300. In another embodiment, the plurality of hollow tines 225 may be angled from about 70° to about 110° from the longitudinal axis 300, alternatively from about 80° to about 100° from the longitudinal axis 300, alternatively from about 85° to about 95° from the longitudinal axis 300, and alternatively at about 90° from the longitudinal axis 300. The angle measurement may be calculated counterclockwise from the upper portion of the longitudinal axis 300 shown in FIG. 4. In an embodiment, one or more of the tines may be angled differently than the other tines.

a. Face

Now referring to FIGS. 2 and 2A, the plurality of hollow tines 225 may each comprise a face 226 located distally from the body portion 220 of the extended multi-tip actuator 200. As described above, the applicator assembly 100 may further comprise a longitudinal axis 300 therethrough. In one embodiment, each face 226 is angled from about 10° to about 30° from the longitudinal axis 300, alternatively from about 15° to about 25° from the longitudinal axis 300, alternatively from about 20° to about 25° from the longitudinal axis 300, and alternatively at about 23° from the longitudinal axis 300. In another embodiment, each face 226 is angled from about 35° to about 55° from the longitudinal axis 300, alternatively from about 40° to about 50° from the longitudinal axis 300, alternatively from about 43° to about 48° from the longitudinal axis 300, and alternatively at about 45° from the longitudinal axis 300. The face 226 angle measurement may be calculated counterclockwise from the upper portion of the longitudinal axis 300 shown in FIG. 4.

b. Aperture

Still referring to FIGS. 2 and 2A, the plurality of hollow tines 225 each may comprise an aperture 227 in fluid communication with the container 150. The aperture 227 may be of any shape, including but not limited to, circular or square.

In one embodiment, each aperture 227 may have a diameter of from about 0.1 mm to about 5 mm. In another embodiment, each aperture 227 may have a diameter of from about 0.2 mm to about 2 mm. In yet another embodiment, each aperture 227 may have a diameter of from about 0.5 mm to about 1.5 mm.

c. Width of Distribution

Now referring to FIG. 3, the plurality of hollow tines 225 may have a width of distribution 400 of from about 1 cm to about 7 cm, alternatively from about 1.5 cm to about 4 cm. and alternatively from about 2 cm to about 3 cm. The width of distribution 400 is the distance between the two external apertures 227 of the plurality of hollow tines 225.

d. Gap

Still referring to FIG. 3, the plurality of hollow tines 225 may also be oriented so that each tine is spaced apart from an adjacent tine by a gap 410 having a width. In one embodiment, the gaps 410 may be of any width capable of letting hair comb through. In one embodiment, the widths 410 may be from about 2 mm to about 20 mm, alternatively from about 3 mm to about 15 mm, alternatively from about 4 mm to about 12 mm, and alternatively from about 4 mm to about 7 mm.

### C. Engine

The applicator assembly 100 may also comprise an engine for delivering a composition from the container 150 through the extended multi-tip actuator 200. The engine may be of any type suitable for dispensing a composition from the container 150, including but not limited to a mechanical pump, aerosol, or squeezing. In one embodiment, the engine may be powered by any means capable of delivering electricity.

In one embodiment, the engine may dispense from about 0.05 mL to about 4 mL of a composition per complete stroke. In another embodiment, the engine may dispense from about 0.1 mL to about 1 mL of the composition per complete stroke. In yet another embodiment, the engine may dispense from about 0.3 mL to about 0.5 mL per complete stroke.

In another embodiment, the engine may deliver an unmetered dose. In this embodiment, the consumer may control the dosage delivered by the applicator assembly 100 by deciding, for example, how long to hold down a button.

### D. Optional Composition

The applicator assembly 100 may further comprise a composition. The composition may be a rinse-off product or a leave-on product, and can be formulated in a wide variety of product forms, including but not limited to liquids, foams, creams, gels, emulsions, powders, and mousses.

In one embodiment, the composition may have a neat viscosity of from about 2,000 cps to about 45,000 cps, alternatively from about 9,000 cps to about 25,000 cps, alternatively from about 9,000 cps to about 12,000 cps, alternatively from about 20,000 cps to about 25,000 cps, alternatively from about 3,000 to about 10,000 cps, and alternatively from about 5,000 to about 8,000 cps.

The neat viscosity of the composition is determined by measuring the viscosity of the composition at a shear rate of 2¹/sec. Scientifically, neat viscosity is the ratio of shear stress to shear rate. Neat viscosity of the composition can be measured with a rheometer. A TA Instrument AR2000 may be used to measure the shear stress curve of the composition.

In one embodiment, the composition may be of any type suitable for application to human skin, including the scalp. In another embodiment, the composition may be of any type suitable for application to pet skin including the roots of the pet hair. In yet another embodiment, the composition may be of any type suitable for application to fabric and/or carpet.

In one embodiment, the composition may comprise one or more components known for use in scalp/hair care or personal care products, provided that the additional components do not otherwise unduly impair product stability, aesthetics, or performance. Such optional ingredients are most typically those described in reference books such as the CTFA Cosmetic Ingredient Handbook, Second Edition, The Cosmetic, Toiletries, and Fragrance Association, Inc. 1988, 1992.

Non-limiting examples of components for use in the composition include conditioning agents (e.g., silicones, hydrocarbon oils, fatty esters), natural cationic deposition polymers, synthetic cationic deposition polymers, anti-dandruff agents, particles, particulate tapioca starch, suspending agents, paraffinic hydrocarbons, propellants, viscosity modifiers, dyes, non-volatile solvents or diluents (water-soluble and water-insoluble), pearlescent aids, foam boosters, surfactants or nonionic cosurfactants, pediculocides, pH adjusting agents, perfumes, preservatives, proteins, skin active agents, sunscreens, UV absorbers, and vitamins.

### 1. Conditioning Agent

In one embodiment, the composition may comprise one or more conditioning agents. Conditioning agents include materials that are used to give a particular conditioning benefit to hair and/or scalp. The conditioning agents that may be useful in the composition typically comprise a water-insoluble, water-dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix.

One or more conditioning agents may be present from about 0.01 wt % to about 10 wt %, alternatively from about 0.1 wt % to about 8 wt %, and alternatively from about 0.2 wt % to about 4 wt %, by weight of the composition.

a. Silicones

The conditioning agent of the composition may be an insoluble silicone conditioning agent. The silicone conditioning agent particles may comprise volatile silicone, non-volatile silicone, or combinations thereof. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

The concentration of the silicone conditioning agent may range from about 0.01% to about 10%, by weight of the composition, alternatively from about 0.1% to about 8%, alternatively from about 0.1% to about 5%, and alternatively from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609, which are incorporated herein by reference. The silicone conditioning agents for use in the composition may have a viscosity, as measured at 25 Â° C., from about 20 to about 2,000,000 centistokes (“csk”), alternatively from about 1,000 to about 1,800,000 csk, alternatively from about 50,000 to about 1,500,000 csk, and alternatively from about 100,000 to about 1,500,000 csk.

The dispersed silicone conditioning agent particles typically have a volume average particle diameter ranging from about 0.01 micrometer to about 50 micrometer. For small particle application to hair, the volume average particle diameters typically range from about 0.01 micrometer to about 4 micrometer, alternatively from about 0.01 micrometer to about 2 micrometer, and alternatively from about 0.01 micrometer to about 0.5 micrometer. For larger particle application to hair, the volume average particle diameters typically range from about 5 micrometer to about 125 micrometer, alternatively from about 10 micrometer to about 90 micrometer, alternatively from about 15 micrometer to about 70 micrometer, and alternatively from about 20 micrometer to about 50 micrometer.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

i. Silicone Oils

Silicone fluids include silicone oils, which are flowable silicone materials having a viscosity, as measured at 25° C., less than 1,000,000 csk, alternatively from about 5 csk to about 1,000,000 csk, and alternatively from about 100 csk to about 600,000 csk. Suitable silicone oils for use in the composition include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

Silicone oils include polyalkyl or polyaryl siloxanes which conform to the following Formula (I):

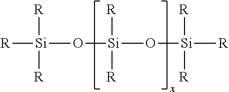

wherein R is aliphatic, in some embodiments alkyl, alkenyl, or aryl, R can be substituted or unsubstituted, and x is an integer from 1 to about 8,000. Suitable R groups for use in the compositions include, but are not limited to: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Possible alkyl and alkenyl substituents include C₁ to C₅ alkyls and alkenyls, alternativelyfrom C₁ to C₄, and alternatively from C₁ to C₂. The aliphatic portions of other alkyl-, alkenyl-, or alkynyl-containing groups (such as alkoxy, alkaryl, and alkamino) can be straight or branched chains, and may be from C₁ to C₅, alternatively from C₁ to C₄, alternatively from C₁ to C₃, and alternatively from C₁ to C₂. As discussed above, the R substituents can also contain amino functionalities (e.g. alkamino groups), which can be primary, secondary or tertiary amines or quaternary ammonium. These include mono-, di-and tri-alkylamino and alkoxyamino groups, wherein the aliphatic portion chain protrusion length may be as described herein.

ii. Amino and Cationic Silicones

Cationic silicone fluids suitable for use in the composition include, but are not limited to, those which conform to the general formula (II):

(R¹)ₐG₃₋ₐ-Si—(—OSiG₂)ₙ-(—OSiG_{b}(R¹)_{2-b})ₘ—O—SiG₃₋ₐ(R¹)ₐ

wherein G is hydrogen, phenyl, hydroxy, or C₁-C₈ alkyl, in some embodiments, methyl; a is 0 or an integer having a value from 1 to 3; b is 0 or 1; n is a number from 0 to 1,999, alternatively from 49 to 499; m is an integer from 1 to 2,000, alternatively from 1 to 10; the sum of n and m is a number from 1 to 2,000, alternatively from 50 to 500; R¹ is a monovalent radical conforming to the general formula CqH_{2q}L, wherein q is an integer having a value from 2 to 8 and L is selected from the following groups:

—N(R²)CH₂—CH₂—N(R²)₂

—N(R²)₂

—N(R²)₃A⁻

—N(R²)CH₂—CH₂—NR²H₂A⁻

wherein R² is hydrogen, phenyl, benzyl, or a saturated hydrocarbon radical, in some embodiments an alkyl radical from about C₁ to about C₂₀, and A⁻ is a halide ion.

In one embodiment, the cationic silicone corresponding to formula (II) is the polymer known as “trimethylsilylamodimethicone”, which is shown below in formula (III):

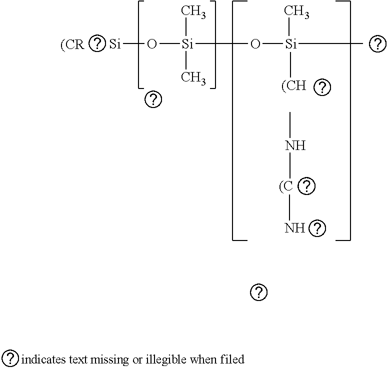

Other silicone cationic polymers which may be used in the composition are represented by the general formula (IV):

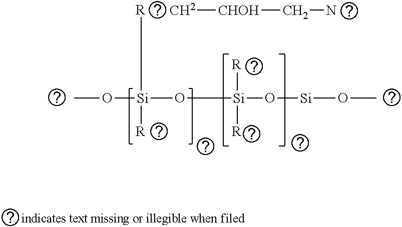

wherein R³ is a monovalent hydrocarbon radical from C₁ to C₁₈, in some embodiments an alkyl or alkenyl radical, such as methyl; R₄ is a hydrocarbon radical, in some embodiments a C₁ to C₁₈ alkylene radical or a C₁₀ to C₁₈ alkyleneoxy radical, alternatively a C₁ to C₈ alkyleneoxy radical; Q⁻ is a halide ion, in some embodiments chloride; r is an average statistical value from 2 to 20, in some embodiments from 2 to 8; s is an average statistical value from 20 to 200, in some embodiments from 20 to 50. One polymer of this class is known as UCARE SILICONE ALE 56®, available from Union Carbide.

iii. Silicone Gums

Other silicone fluids suitable for use in the composition may be insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76, all of which are incorporated herein by reference. Specific non-limiting examples of silicone gums for use in the hair care include polydimethylsiloxane, (polydimethylsiloxane)(methylvinylsiloxane)copolymer, poly(dimethylsiloxane)(diphenyl siloxane)(methylvinylsiloxane)copolymer and mixtures thereof.

iv. High Refractive Index Silicones

Other non-volatile, insoluble silicone fluid conditioning agents that are suitable for use in the composition are those known as “high refractive index silicones,” having a refractive index of at least about 1.46, alternatively at least about 1.48, alternatively at least about 1.52, and alternatively at least about 1.55. The refractive index of the polysiloxane fluid will generally be less than about 1.70, typically less than about 1.60. In this context, polysiloxane “fluid” includes oils as well as gums. The high refractive index polysiloxane fluid includes those represented by general Formula (I) above, as well as cyclic polysiloxanes such as those represented by Formula (V) below:

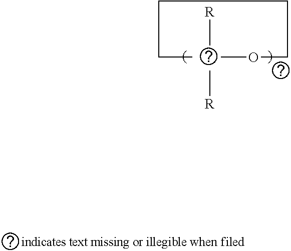

wherein R is as defined above, and n is a number from about 3 to about 7, alternatively from about 3 to about 5.

The high refractive index polysiloxane fluids contain an amount of aryl-containing R substituents sufficient to increase the refractive index to the desired level, which is described herein. Additionally, R and n may be selected so that the material is non-volatile.

Aryl-containing substituents include those which contain alicyclic and heterocyclic five and six member aryl rings and those which contain fused five or six member rings. The aryl rings themselves can be substituted or unsubstituted.

Generally, the high refractive index polysiloxane fluids will have a degree of aryl-containing substituents of at least about 15%, alternatively at least about 20%, alternatively at least about 25%, alternatively at least about 35%, and alternatively at least about 50%. Typically, the degree of aryl substitution will be less than about 90%, more generally less than about 85%, alternatively from about 55% to about 80%. In some embodiments, the high refractive index polysiloxane fluids have a combination of phenyl or phenyl derivative substituents, with alkyl substituents, in some embodiments C₁-C₄ alkyl, hydroxy, or C₁-C₄ alkylamino (especially—R⁴NHR⁵NH2 wherein each R⁴ and R⁵ independently is a C₁-C₃ alkyl, alkenyl, and/or alkoxy).

When high refractive index silicones are used in the composition, they may be used in composition with a spreading agent, such as a silicone resin or a surfactant, to reduce the surface tension by a sufficient amount to enhance spreading and thereby enhance the glossiness (subsequent to drying) of hair treated with the compositions.

Silicone fluids suitable for use in the composition are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984), all of which are incorporated herein by reference.

v. Silicone Resins

Silicone resins may be included in composition. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra-or tetra-functional unit SiO₂. Primes of the unit symbols (e.g. M′, D′, T′, and Q′) denote substituents other than methyl, and must be specifically defined for each occurrence.

Silicone resins for use in the composition may include, but are not limited to MQ, MT, MTQ, MDT and MDTQ resins. Methyl is a possible silicone substituent. In some embodiments, silicone resins are MQ resins, wherein the M:Q ratio is from about 0.5:1.0 to about 1.5:1.0 and the average molecular weight of the silicone resin is from about 1000 to about 10,000.

The weight ratio of the non-volatile silicone fluid, having refractive index below 1.46, to the silicone resin component, when used, may be from about 4:1 to about 400:1, alternatively from about 9:1 to about 200:1, and alternatively from about 19:1 to about 100:1, particularly when the silicone fluid component is a polydimethylsiloxane fluid or a mixture of polydimethylsiloxane fluid and polydimethylsiloxane gum as described herein. Insofar as the silicone resin forms a part of the same phase in the compositions hereof as the silicone fluid, i.e. the conditioning active, the sum of the fluid and resin should be included in determining the level of silicone conditioning agent in the composition.

b. Organic Conditioning Oils

The conditioning agent of the composition may also comprise at least one organic conditioning oil, either alone or in combination with other conditioning agents, such as the silicones described above.

i. Hydrocarbon Oils

Suitable organic conditioning oils for use as conditioning agents in the composition may include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils may be from about C₁₂ to about C₁₉. Branched chain hydrocarbon oils, including hydrocarbon polymers, typically will contain more than 19 carbon atoms.

ii. Polyolefins

Organic conditioning oils for use in the composition may also include liquid polyolefins, alternatively liquid poly-α-olefins, alternatively hydrogenated liquid poly-α-olefins. Polyolefins for use herein are prepared by polymerization of C₄ to about C₁₄ olefenic monomers, in some embodiments from about C₆ to about C₁₂.

iii. Fatty Esters

Other suitable organic conditioning oils for use as the conditioning agent in the composition may include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

iv. Fluorinated Conditioning Compounds

Fluorinated compounds suitable for delivering conditioning to hair or skin as organic conditioning oils include perfluoropolyethers, perfluorinated olefins, fluorine based specialty polymers that may be in a fluid or elastomer form similar to the silicone fluids previously described, and perfluorinated dimethicones.

v. Fatty Alcohols

Other suitable organic conditioning oils for use in the composition may include, but are not limited to, fatty alcohols having at least about 10 carbon atoms, alternativelyfrom about 10 to about 22 carbon atoms, and alternatively from about 12 to about 16 carbon atoms.

vi. Alkyl Glucosides and Alkyl Glucoside Derivatives

Suitable organic conditioning oils for use in the composition may include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

c. Other Conditioning Agents

i. Quaternary Ammonium Compounds

Suitable quaternary ammonium compounds for use as conditioning agents in the composition may include, but are not limited to, hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

ii. Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof.

iii. Cationic Deposition Polymers

The composition may further comprise a cationic deposition polymer. Any known natural or synthetic cationic deposition polymer can be used herein. Examples include those polymers disclosed in U.S. Pat. No. 6,649,155; U.S. Patent Application Publication Nos. 2008/0317698; 2008/0206355; and 2006/0099167, which are incorporated herein by reference in their entirety.

The cationic deposition polymer may be included in the composition at a level from about 0.01 wt % to about 2 wt %, in one embodiment from about 1.5 wt % to about 1.9 wt %, in another embodiment from about 1.8 wt % to about 2.0 wt %, in view of providing the benefits of the composition.

The cationic deposition polymer may be a water soluble polymer with a charge density from about 0.5 milliequivalents per gram to about 12 milliequivalents per gram. The cationic deposition polymer used in the composition may have a molecular weight of about 100,000 Daltons to about 5,000,000 Daltons. The cationic deposition polymer may be a low charge density cationic polymer.

In one embodiment, the cationic deposition polymer is a synthetic cationic deposition polymer. A variety of synthetic cationic deposition polymers can be used including mono- and di-alkyl chain cationic surfactants. In one embodiment, mono-alkyl chain cationic surfactants are chosen including, for example, mono-alkyl quaternary ammonium salts and mono-alkyl amines. In another embodiment, di-alkyl chain cationic surfactants are used and include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, and mixtures thereof.

In another embodiment, the cationic deposition polymer is a naturally derived cationic polymer. The term, “naturally derived cationic polymer” as used herein, refers to cationic deposition polymers which are obtained from natural sources. The natural sources may be polysaccharide polymers. Therefore, the naturally derived cationic polymer may be selected from the group comprising starches, guar, cellulose, Cassia, locust bean, Konjac, Tara, galactomannan, tapioca, and synthetic polymers. In a further embodiment, cationic deposition polymers are selected from Mirapol® 100S (Rhodia), Jaguar® C17, polyDADMAC, Tapioca starch (Akzo), Triquat™, and mixtures thereof.

d. Anionic Emulsifiers

A variety of anionic emulsifiers can be used in the composition as described below. The anionic emulsifiers include, by way of illustrating and not limitation, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, gum arabic, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates.

In addition, anionic emulsifiers that have acrylate functionality may also be used in the composition. Anionic emulsifiers useful herein include, but aren't limited to: poly(meth)acrylic acid; copolymers of (meth)acrylic acids and its (meth)acrylates with C1-22 alkyl, C1-C8 alkyl, butyl; copolymers of (meth)acrylic acids and (meth)acrylamide; Carboxyvinylpolymer; acrylate copolymers such as Acrylate/C10-30 alkyl acrylate crosspolymer, Acrylic acid/vinyl ester copolymer/Acrylates/Vinyl Isodecanoate crosspolymer, Acrylates/Palmeth-25 Acrylate copolymer, Acrylate/Steareth-20 Itaconate copolymer, and Acrylate/Celeth-20 Itaconate copolymer; Polystyrene sulphonate, copolymers of methacrylic acid and acrylamidomethylpropane sulfonic acid, and copolymers of acrylic acid and acrylamidomethylpropane sulfonic acid; carboxymethycellulose; carboxy guar; copolymers of ethylene and maleic acid; and acrylate silicone polymer. Neutralizing agents may be included to neutralize the anionic emulsifiers herein. Non-limiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof. Commercially available anionic emulsifiers include, for example, Carbomer supplied from Noveon under the tradename Carbopol 981 and Carbopol 980; Acrylates/C10-30 Alkyl Acrylate Crosspolymer having tradenames Pemulen TR-1, Pemulen TR-2, Carbopol 1342, Carbopol 1382, and Carbopol ETD 2020, all available from Noveon; sodium carboxymethylcellulose supplied from Hercules as CMC series; and Acrylate copolymer having a tradename Capigel supplied from Seppic. In another embodiment, anionic emulsifiers are carboxymethylcelluloses.

### 2. Benefit Agents

In an embodiment, the composition further comprises one or more additional benefit agents. The benefit agents comprise a material selected from the group consisting of anti-dandruff agents, vitamins, lipid soluble vitamins, chelants, perfumes, brighteners, enzymes, sensates, attractants, anti-bacterial agents, dyes, pigments, bleaches, hops, resorcinol, caffeine, cleaning agents, and mixtures thereof.

a. Vitamin B₃ Compounds

The composition may include a vitamin B3 compound. In one embodiment, the vitamin B3 compound is niacinamide. Vitamin B3 compounds may be useful for regulating skin conditions, as described in U.S. Pat. No. 5,939,082. In some embodiments, the composition may comprise from about 0.1% to about 25% of a vitamin B3 compound, in another embodiment from about 0.5% to about 15% of a vitamin B3 compound, and in yet another embodiment from about 3.5% to about 7.5% of a vitamin B3 compound. As used herein, “vitamin B3 compound” means a one or more compounds having the formula:

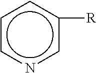

wherein R is —CONH₂ (i.e., niacinamide), —COOH (i.e., nicotinic acid) or —CH2OH (i.e., nicotinyl alcohol); derivatives thereof; mixtures thereof; and salts of any of the foregoing.

Exemplary derivatives of the foregoing vitamin B3 compounds include nicotinic acid esters, including non-vasodilating esters of nicotinic acid (e.g, tocopherol nicotinate, myristyl nicotinate), nicotinyl amino acids, nicotinyl alcohol esters of carboxylic acids, nicotinic acid N-oxide and niacinamide N-oxide. Additional exemplary derivatives of vitamin B3 compounds are set forth in U.S. patent application Ser. No. 11/897084, which is incorporated herein by reference.

b. Alcohol

In one embodiment, the composition may comprise an alcohol. Alcohol may be used for faster drying and skin penetration of the composition. In a particular embodiment, the composition comprises from about 10% to about 90% alcohol, alternatively from about 15% to about 75% alcohol, or alternatively from about 25% to about 50% alcohol. Any suitable alcohol, such as ethanol, can be used.

c. Anti-Dandruff Agent

In one embodiment, the composition may comprise an anti-dandruff agent, which may be an anti-dandruff active particulate. Such anti-dandruff particulate should be physically and chemically compatible with the components of the composition, and should not otherwise unduly impair product stability, aesthetics or performance.

In an embodiment, the anti-dandruff agent may be selected from the group consisting of: pyridinethione salts; azoles, such as ketoconazole, econazole, and elubiol; selenium sulphide; particulate sulfur; keratolytic agents such as salicylic acid; and mixtures thereof.

Pyridinethione salts may be suitable anti-dandruff active particulates. In an embodiment, the anti-dandruff active may be a 1-hydroxy-2-pyridinethione salt and is in particulate form. In an embodiment, the concentration of pyridinethione anti-dandruff particulate ranges from about 0.01 wt % to about 5 wt %, or from about 0.1 wt % to about 3 wt %, or from about 0.1 wt % to about 2 wt %. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

In an embodiment, in addition to the anti-dandruff active selected from polyvalent metal salts of pyrithione, the composition may further comprise one or more anti-fungal and/or anti-microbial actives. In an embodiment, the anti-microbial active is selected from the group consisting of: coal tar, sulfur, charcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the composition, the azole anti-microbial active may be included in an amount of from about 0.01 wt % to about 5 wt %, or from about 0.1 wt % to about 3 wt %, or from about 0.3 wt % to about 2 wt %. In an embodiment, the azole anti-microbial active is ketoconazole. In an embodiment, the sole anti-microbial active is ketoconazole.

Embodiments of the composition may also comprise a combination of anti-microbial actives. In an embodiment, the combination of anti-microbial actives is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the composition comprises an effective amount of a zinc-containing layered material. In an embodiment, the composition comprises from about 0.001 wt % to about 10 wt %, or from about 0.01 wt % to about 7 wt %, or from about 0.1 wt % to about 5 wt % of a zinc-containing layered material, by total weight of the composition.

Zinc-containing layered materials may be those with crystal growth primarily occurring in two dimensions. It is conventional to describe layered structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+}A^{m-}_{x/m}·nH₂O wherein some or all of the divalent ions (M²⁺) are zinc ions (Crepaldi, E L, Pava, P C, Tronto, J, Valim, J B J. Colloid Interfac. Sci. 2002, 248, 429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺Aⁿ⁻_{(1=3/y)/n}·nH₂O where the two metal ions (M²⁺) may be the same or different. If they are the same and represented by zinc, the formula simplifies to [Zn₁₊ₓ(OH)₂]^{2x+}2x A⁻·nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replace the monovalent anion. These materials can also be formed in situ in a composition or in or during a production process.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the anti-dandruff active is at least about 1 microgram/cm². The on-scalp deposition of the anti-dandruff active is important in view of ensuring that the anti-dandruff active reaches the scalp where it is able to perform its function. In an embodiment, the deposition of the anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction composition is added and agitated prior to recovery and analytical determination of anti-dandruff active content by conventional methodology, such as HPLC.

d. Fatty Alcohol Gel Network

Embodiments of the composition may also comprise fatty alcohol gel networks, which have been used for years in cosmetic creams and hair conditioners. These gel networks are formed by combining fatty alcohols and surfactants in the ratio of about 1:1 to about 40:1 (alternatively from about 2:1 to about 20:1, and alternatively from about 3:1 to about 10:1). The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. The gel network contributes a stabilizing benefit to cosmetic creams and hair conditioners. In addition, they deliver conditioned feel benefits for hair conditioners.

Thus according to an embodiment, the fatty alcohol is included in the fatty alcohol gel network at a level by weight of from about 0.05 wt % to about 14 wt %. For example, the fatty alcohol may be present in an amount ranging from about 1 wt % to about 10 wt %, and alternatively from about 6 wt % to about 8 wt %.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof. Mixtures of cetyl and stearyl alcohol in a ratio of from about 20:80 to about 80:20, are suitable.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof. Mixtures of cetyl and stearyl alcohol in a ratio of from about 20:80 to about 80:20, are suitable.

### E. Method for Dispensing

The applicator assembly 100 described above may also be used in a method for dispensing a composition. The method for dispensing a composition may comprise providing the applicator assembly 100 described above, disposing a composition in the applicator assembly 100, and activating the applicator assembly 100. In one embodiment, the activating of the applicator assembly 100 may include actuating a mechanical pump,an aerosol container, or a squeeze container.

In one embodiment, the method may be used for dispensing the composition from the applicator assembly directly onto the scalp.

In another embodiment, the method may also be used for improving the efficacy of a composition after being delivered to the scalp.

### F. Data

Referring to Table 1, 30 consumers ages 22 to 55 were asked to rate the following scalp applicators. The spray applicator is a common in-store applicator used for applying compositions to the scalp. The multi-tine applicator is an embodiment of the present invention used for applying compositions to the scalp. The multi-tine applicator has three tines. The consumers were asked to rate the scalp applicators on a scale of 1 to 10, 10 being most useful for scalp application, and 1 being most useful for hair application. The mean values were then calculated and listed in Table 1. Using the Student T method at a 95% confidence level, there was a statistically significant difference in the consumers' preference for the multi-tine applicator for being most useful for scalp application.

TABLE 1
Usefulness for Scalp Application

| Applicator | Mean |
|---|---|
| Multi-Tine | 7.67 |
| Spray | 2.83 |

Referring to Table 2, 30 consumers ages 22 to 55 were asked to rate the following scalp applicators. The spray applicator is a common in-store applicator used for applying compositions to the scalp. The multi-tine applicator is an embodiment of the present invention used for applying compositions to the scalp. The multi-tine applicator has three tines. The consumers were asked to rate the scalp applicators on a scale of 1 to 10, 10 being easiest to apply to the composition to the scalp, and 1 being hardest to apply the composition to the scalp. The mean values were then calculated and listed in Table 2. Using the Student T method at a 95% confidence level, there was a statistically significant difference in the consumers' preference for the multi-tine applicator for being easiest to apply the composition to the scalp.

TABLE 2
Easy to Apply

| Applicator | Mean |
|---|---|
| Multi-Tine | 7.13 |
| Spray | 2.67 |

Referring to Table 3, 30 consumers ages 22 to 55 were asked to rate the following scalp applicators. The spray applicator is a common in-store applicator used for applying compositions to the scalp. The multi-tine applicator is an embodiment of the present invention used for applying compositions to the scalp. The multi-tine applicator has three tines. The consumers were asked to rate the scalp applicators on a scale of 1 to 10, 10 being most efficient for applying the composition to the scalp, and 1 being least efficient for applying the composition to the scalp. The mean values were then calculated and listed in Table 3. Using the Student T method at a 95% confidence level, there was a statistically significant difference in the consumers' preference for the multi-tine applicator for being most efficient for applying the composition to the scalp.

TABLE 3
Efficiency

| Applicator | Mean |
|---|---|
| Multi-Tine | 7.00 |
| Spray | 3.43 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A method for delivering a composition to the scalp comprising:
a. providing a composition in an applicator assembly, the applicator assembly comprising:
i. a container for holding the composition;
ii. an extended tip actuator in fluid communication with the container, the extended tip actuator comprising:
1. a base portion configured to fluidly connect the extended tip actuator to the container; and
2. a body portion configured to fluidly connect the base portion to a plurality of hollow tines, wherein the tines each comprise a face located distally from the body portion, wherein the tines each comprise an aperture in fluid communication with the container, and wherein the tines each have a protrusion length of from about 0.5 mm to about 100 mm; and
iii. an engine for delivering the composition from the container through the extended tip actuator; and
b. dispensing the composition from the applicator assembly directly onto the scalp.

2) The method of claim 1, wherein the tines each have a protrusion length of from about 10 mm to about 60 mm.

3) The method of claim 1, wherein the tines each have a protrusion length of from about 20 mm to about 50 mm.

4) The method of claim 1, wherein each aperture has a diameter of from about 0.1 mm to about 5 mm.

5) The method of claim 4, wherein each aperture has a diameter of from about 0.2 mm to about 2 mm.

6) The method of claim 1, wherein the engine is a mechanical pump.

7) The method of claim 1, wherein the engine dispenses from about 0.05 mL to about 4 mL per complete stroke.

8) The method of claim 1, wherein the plurality of hollow tines comprises three tines.

9) The method of claim 1, wherein the tines are positioned vertically.

10) The method of claim 1, wherein the applicator assembly further comprises a longitudinal axis therethrough, and the tines are angled from about 20° to about 100° from the longitudinal axis.

11) The method of claim 1, wherein the applicator assembly further comprises a longitudinal axis therethrough, and the tines are angled from about 25° to about 70° from the longitudinal axis.

12) The method of claim 1, wherein the applicator assembly further comprises a longitudinal axis therethrough, and wherein each face is angled from about 30° to about 60° from the longitudinal axis.

13) The method of claim 1, wherein the applicator assembly further comprises a longitudinal axis therethrough, and wherein each face is angled from about 35° to about 55° from the longitudinal axis.

14) The method of claim 1, wherein the tines have a width of distribution of from about 1 cm to about 7 cm.

15) The method of claim 1, wherein each tine is spaced apart from an adjacent tine by a gap having a width of from about 2 cm to about 4 cm.

16) The method of claim 1, wherein the applicator assembly further comprises a composition comprising from about 0.1% to about 25% niacinamide.

17) The method of claim 1, wherein the composition comprises from about 10% to about 90% alcohol.

18) The method of claim 1, wherein the composition comprises an anti-dandruff agent.

19) An applicator assembly for delivering a composition to the scalp comprising:
a. a container for holding the composition;
b. an extended multi-tip actuator in fluid communication with the container, the extended tip actuator comprising:
i. a base portion configured to fluidly connect the extended multi-tip actuator to the container; and
ii. a body portion configured to fluidly connect the base portion to a plurality of hollow tines, wherein the tines each comprise a face located distally from the body portion, wherein the tines each comprise an aperture in fluid communication with the container, and wherein the tines each have a protrusion length of from about 0.5 mm to about 100 mm; and
c. an engine for delivering the composition from the container through the extended multi-tip actuator to the scalp.

20) The method of claim 19, wherein the composition comprises from about 0.1% to about 25% niacinamide.
